(19) United States
(12) Patent Application Publication
(10) Pub. No.: US 2009/0287167 A1
(43) Pub. Date: Nov. 19, 2009

(54) Y-PORT DEVICE ELIMINATING ABBERANT CURRENTS

(75) Inventors: Bryan G. Davis, Sandy, UT (US); Bart D. Peterson, Farmington, UT (US)

Correspondence Address: David W. Highet, VP & Chief IP Counsel;Becton, Dickinson and Company, (Kirton & McConkie), 1 Becton Drive, MC 110, Franklin Lakes, NJ, 07417-1880, US

(73) Assignee: BECTON, DICKINSON AND COMPANY, Franklin Lakes, NJ (US)

(21) Appl. No.: 12/120,431

(22) Filed: May 14, 2008

(51) Int. Cl. A61M 25/18

(52) U.S. Cl. 604/284

(57) ABSTRACT

A y-port device including a valve positioned proximally to the fluid channel thereby eliminating aberrant currents and dead space within the y-port device. The valve is a septum and penetrable by a probe. The valve is positioned such that the external end may be cleaned and/or sanitized prior to penetration by a probe and the internal end abuts the fluid channel of the device such that dead space is eliminated between the flow path and the internal end of the valve. The y-port device ensures that an entire infused bolus is flushed from the interior of the y-port and into the desired vascular system.

### BACKGROUND OF THE INVENTION

The present disclosure relates to infusion systems generally and specifically to the use of a y-port device during intravenous therapy.

Intravenous therapy is one of the most common health care procedures. Hospitalized, home care, and other patients receive fluids, pharmaceuticals, and blood products via a vascular access device inserted into the vascular system. Infusion therapy may be used to treat an infection, provide anesthesia or analgesia, provide nutritional support, treat cancerous growths, maintain blood pressure and heart rhythm, or many other clinically significant uses.

Intravenous therapy is facilitated by vascular access devices located outside the vascular system of a patient (extravascular devices). Extravascular devices that may access a patient's peripheral or central vasculature, either directly or indirectly include closed access devices, such as the BD Q-SYTE closed Luer access device of Becton, Dickinson and Company; syringes; split access devices; catheters; and intravenous (IV) fluid chambers. A vascular device may be indwelling for short term (days), moderate term (weeks), or long term (months to years). A vascular access device may be used for continuous infusion therapy or for intermittent therapy.

A common vascular access device is a plastic catheter that is inserted into a patient's vein. The catheter length may vary from a few centimeters for peripheral access to many centimeters for central access. The catheter may be inserted transcutaneously or may be surgically implanted beneath the patient's skin. The catheter, or any other extravascular device attached thereto, may have a single lumen or multiple lumens for infusion of many fluids simultaneously. For example, a catheter may be attached to a section of tubing wherein the section of tubing is also attached to an IV fluid chamber. This configuration allows the patient to receive fluids through the catheter without having the fluid chamber located near the catheter.

A vascular access device is commonly incorporated into an infusion system. For example, a vascular access device may be attached to a first end of a section of tubing wherein the second end of the tubing is attached to an IV fluid chamber. The infusion system may include an access port. For example, the access port may be incorporated into the middle portion of the section of tubing thereby allowing for multiple, concurrent therapies using the same vascular access device. For example, if a first therapeutic agent is contained in an IV fluid chamber and being administered to a patient via a catheter, a second therapeutic agent may be administered simultaneously through the access port of the catheter without interrupting the administration of the first therapeutic agent. One commonly used access port is a y-port.

A y-port is commonly coupled to a vascular access device via a section of tubing. The y-port is generally adapted to receive a pair of connector tips through which fluids and/or therapeutic agents may be administered. Typically, one of the connector tips is attached to a length of tubing to which is also connected an IV fluid chamber. The remaining access port is typically designed to allow access for a sharp needle or a blunt probe. This is accomplished by inserting an accessible plug or valve into the access port. For example, the plug or valve may be a split septum or a puncturable septum. A split septum is a solid or semi-solid plug that has been cut through the center such that an access channel is created. This access channel is opened only by forcing a correctly sized object through the channel and into the interior of the y-port. A puncturable septum is a solid or semi-solid plug that is capable of being punctured by a sharp needle. Both types of septum are typically designed to close upon withdrawal of the probe such that the fluid within the interior of the y-port is unable to exit the plug or valve.

Traditional placement of the plug or valve in a y-port creates undesirable dead spaces within the interior of the y-port. These dead spaces are created by positioning the valve or plug within the opening of the access port such that the outer surface of the valve or plug is near the opening of the access port and the inner surface of the valve or plug is located at a position recessed from the fluid channel of the y-port. This recessed position creates a cove within the interior of the y-port where aberrant currents are formed causing the rate of flow to decrease, backflows to occur and fluid to be trapped and/or concentrated. This effect is undesirable for several reasons.

The effect is undesirable because if medication is trapped in the dead space, the medication will not be delivered to the patient as expected. The obvious drawback of this effect is that undelivered medication is unable to provide an intended benefit to the patient. This means that a patient may suffer due to the inability of the clinician to effectively deliver the medication to the patient. For example, a clinician may administer a first bolus of a medication through the y-port expecting a desired effect. Upon lack of the desired effect, the clinician may administer a second, larger bolus wherein an infused combination of the second larger bolus and the remainder of the first bolus. Additionally, the clinician may choose to administer a second bolus of a different medication wherein the mixing of the second bolus and the remaining first bolus results in an undesired effect in the patient or in the infusion system. Such an effect may be an allergic reaction in the patient or a precipitation of the mediations in the y-port thereby clogging the y-port or clogging the patient's vein resulting in vascular damage.

As medication is trapped in the dead space, the medication may become concentrated. Generally, fluid flows from the IV fluid chamber though the tubing of the infusion system, through the y-port interior, into the vascular access device and into the patient's vascular system. Infusion systems are designed such that the flow of fluid from an IV fluid chamber to a patient's vascular system is continuous and efficient. The cove created by the recessed position of the valve or plug disrupts the continuous and efficient flow of the infusion system by creating aberrant currents, backflows and/or eddies within the interior of the y-port. These disruptions result in a reduced rate of flow within the dead space, thereby preventing the trapped medication from efficiently mixing with the fluid flowing through the y-port.

As a result, the medication becomes concentrated within the dead space. Upon subsequent usage of the y-port, the concentrated medication may be forced into the patient's vascular system with adverse results. For example, when sedating a newborn for a surgical procedure, the limited volume of the neonatal patient's vascular system requires that boluses of medication be highly concentrated thereby minimizing the volume of the bolus. When the highly concentrated bolus is infused via a y-port, only a portion of the desired medication is actually received by the patient while the remainder of the bolus is trapped in the dead space. Following the procedure, as subsequent therapeutic agents are administered to aid the patient's recovery, the stored, highly concentrated medication is forced from the dead space and administered to the patient thereby prolonging the sedated state of the patient.

Therefore, a need exists for systems and methods that eliminate aberrant currents within the y-port device, yet still provide convenient access to the infusion system.

### BRIEF SUMMARY OF THE INVENTION

The present invention has been developed in response to problems and needs in the art that have not been fully resolved by currently available infusion systems, devices, and methods for intravenous therapy. Specifically, the current invention addresses problems in the art associated with aberrant currents present in y-port devices. Dead space, as used in reference to the current invention, is an area within a channel of fluid where the flow of the fluid is diverted and/or the flow rate of the fluid is decreased such that a portion of the fluid becomes stagnant and/or concentrated. These dead spaces may be formed due to recessed areas within the fluid channel thereby creating aberrant currents within the flow path. Thus, these developed systems, devices, and methods provide an infusion system that eliminates dead space thereby ensuring that liquids are infused directly into the flow path of the infusion system and ultimately into the vascular system of the patient.

The y-port device of the present invention may include a first tubular member having a first end and a second end and extending in a generally longitudinal direction. The y-port device may also include a second tubular member intersecting the first tubular member and forming a junction wherein a fluid channel is created between the first and second tubular members. The fluid channel is continuous and generally uniform in diameter such that the dynamics of the fluid flow are uniform throughout the interior of the y-port. The y-port device may also include an access valve or plug through which the infusion system may be accessed. The access valve or plug may include a one-way access valve, such as a split septum or a puncturable septum. For example, a split septum may include a dividing wall wherein the two halves of the wall are biased together such that a barrier is formed. This barrier is penetrable by a correctly sized probe wherein the probe may include a blunt cannula. A puncturable septum may include a membrane that is positioned within the first tubular member so as to form a seal between the exterior and the interior of the y-port. The membrane is capable of being penetrated or punctured by a sharp probe wherein the sharp probe may include a needle. Upon removal of the sharp probe, the walls of the membrane are biased radially inwardly thereby enclosing the access channel created by the sharp probe. Each type of septum may comprise a solid or semi-solid material.

It is also anticipated that the y-port device may include a multi-way access valve such that fluids may be added to or withdrawn from the infusion system. For example, the multi-way access valve may include a flow-stop valve, a ball valve or a multi-turn valve. Additionally, it is anticipated that a non-valve feature may be used in place of an access valve. For example, a plug or cap may be used wherein the plug or cap is placed within the first tubular member and designed so as eliminate any recessed cove between the terminal end of the cap or plug and the flow path.

The valve may be housed within the first tubular member and extend from the first end of the first tubular member to the junction of the first and second tubular members. The access valve terminates in an angle generally corresponding to the angle of the junction between the first and second tubular members. For example, if the junction of the first and second tubular member is at an angle of 120°, then the valve will terminate at an angle of 120°. Thus the inner profile of the second tubular member is maintained by the terminal end of the access valve. In this way, the terminal end of the access valve creates a direct interface with the flow path such that no recessed cove exists between the terminal end of the access valve and the flow path.

The direct interface between the terminal end of the access valve and the flow path ensures that any fluid infused into the infusion system is infused directly into the flow path and ultimately into the vascular system of the patient. The lack of dead space prevents the formation of a concentrated reserve of the infused fluid within the fluid channel. The direct interface of the terminal end of the plug and the flow path ensures that the flow path is continuous in one direction thereby eliminating eddies or fluid pockets in the infusion system where fluid may gather and concentrate.

A method of preventing undesired concentrations of one fluid within a stream of a second fluid may be accomplished by incorporating the valve or plug of the present invention into a desired infusion system. For example, a clinician may select an infusion system for a specific need and incorporate a y-port device that has been modified to include an access valve or plug that eliminates any recessed cove within the interior of the y-port device. Additionally, a clinician may select an infusion system for a specific need and incorporate an access valve or plug into the system thereby eliminating any recessed cove within the infusion system thereby eliminating any potential for undesired concentration of one fluid within a stream of a second fluid.

An infusion system may include a y-port device comprising a valve means wherein the positioning of the terminal end of the valve means eliminates dead space within the interior of the y-port device. The valve means may also be penetrable such that the interior of the y-port device may be accessed through the valve means. The valve means may also be positioned to facilitate direct access into the fluid channel of the y-port device when the valve means is utilized to access the interior of the y-port device.

### BRIEF DESCRIPTION OF THE SEVERAL VIEWS OF THE DRAWINGS

In order that the manner in which the above-recited and other features and advantages of the invention are obtained will be readily understood, a more particular description of the invention briefly described above will be rendered by reference to specific embodiments thereof which are illustrated in the appended drawings. These drawings depict only typical embodiments of the invention and are not therefore to be considered to limit the scope of the invention.

FIG. 1 is a perspective view of the y-port device as incorporated into an infusion system.

FIG. 2 is a cross section view of the y-port device showing the valve and the orientation of the valve with respect to the fluid channel.

FIG. 3 is a partially cut-away perspective view of the y-port device with a split septum and respective probe.

FIG. 4 is a partially cut-away perspective view of the y-port device with a puncturable septum and respective probe.

FIG. 5 is a partially cut-away view of the y-port device with a split septum as penetrated by a probe.

### DETAILED DESCRIPTION OF THE INVENTION

The presently preferred embodiments of the present invention will be best understood by reference to the drawings, wherein like reference numbers indicate identical or functionally similar elements. It will be readily understood that the components of the present invention, as generally described and illustrated in the figures herein, could be arranged and designed in a wide variety of different configurations. Thus, the following more detailed description, as represented in the figures, is not intended to limit the scope of the invention as claimed, but is merely representative of presently preferred embodiments of the invention.

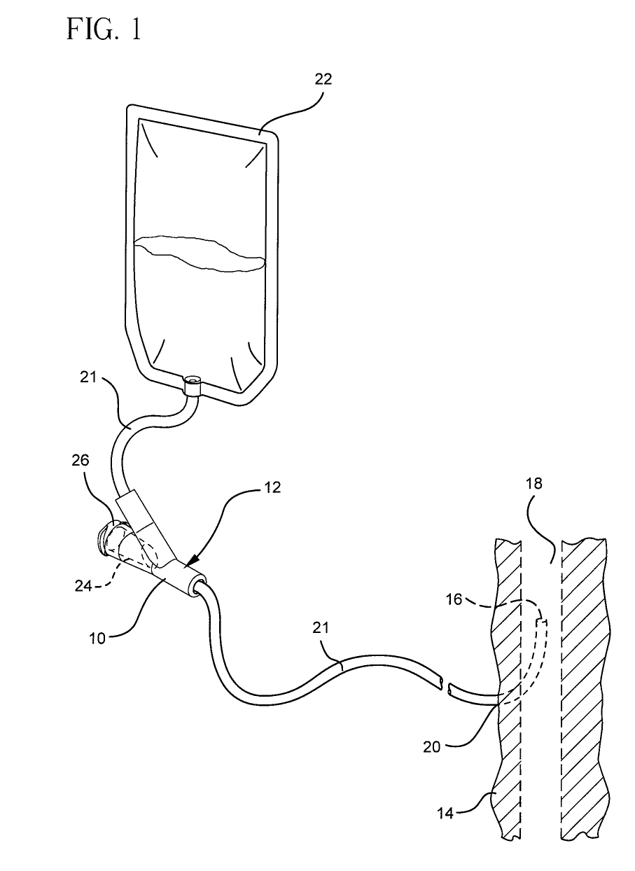

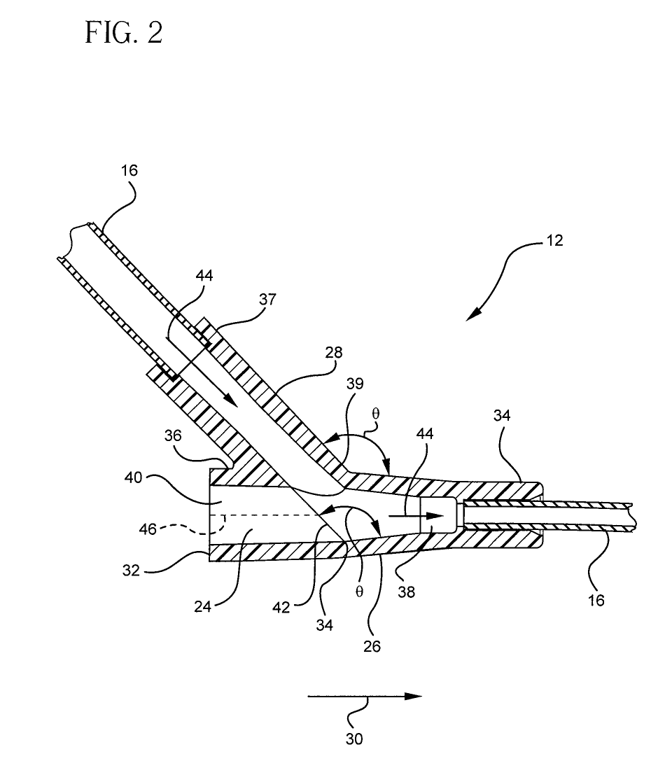

Referring now to FIGS. 1 and 2, a y-port device 10 is illustrated in an infusion system 12 wherein a patient 14 receives intravenous therapy via the insertion of a catheter tube 16 into the patient 14. The infusion system 12 comprises a catheter tube 16, a y-port device 10, and intravenous tubing 21. The infusion system may also include a pre-filled sterile container of fluids 22 or any other source of fluid and/or therapeutic agent. The y-port device 10 provides an access point in the catheter tube 16 thereby allowing a user and/or clinician to access the patient's vascular system 18 without disturbing the catheter insertion site 20 or the pre-filled, sterile container of fluids 22. The y-port device 10 permits access to the catheter tube via a valve 24 as located with a first tubular member 26 of the y-port device 10. The valve 24 may be any valve adaptable to the present invention.

For example, the valve may be a septum, where the septum may be bypassed in order to access the interior of the y-port device. In one embodiment the valve 24 is a split septum 46 wherein the septum 24 is cut in a generally longitudinal direction 30 such that a split 46 is created through the center of the valve 24, this split 46 forming an access channel through the center of the valve 24. The septum split 46 may be biased so as to remain in a closed position until the walls of the split 46 are forced apart by the introduction of a probe 53 into the split 46. The probe 53 may be a blunt cannula 48, such as a male Luer, or any probe-like structure appropriately sized and adapted to access the fluid channel 38 of the infusion system 12 through the valve 24.

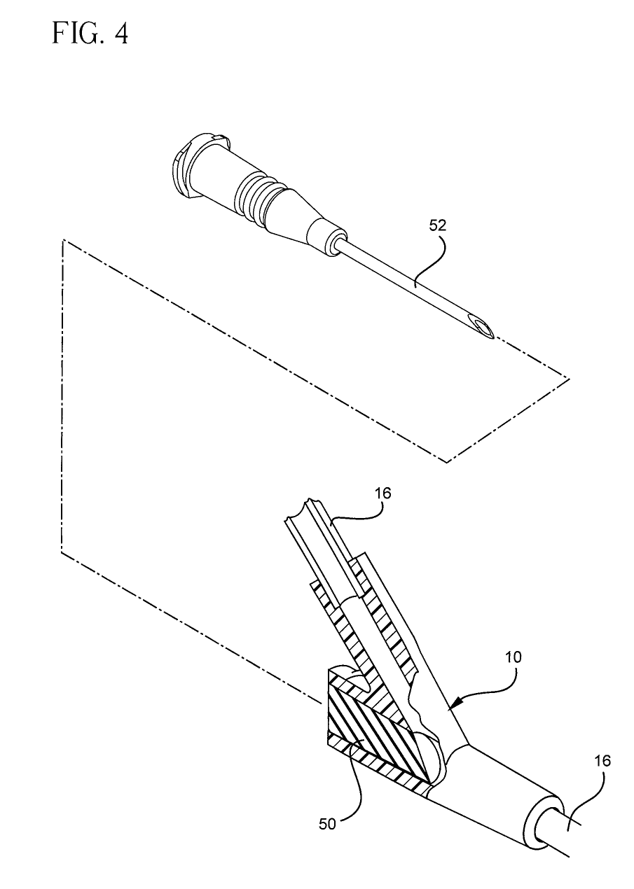

In another embodiment, as illustrated in FIG. 4, the valve 24 is a penetrable membrane 50 wherein the penetrable membrane 50 comprises a solid or semi-solid plug which may be penetrated by a sharp probe. The sharp probe may be a hypodermic needle 52 or any needle-like structure adapted to penetrate the membrane 24 and access the fluid channel 38 of the infusion system 12. In one embodiment, the puncturable membrane 50 comprises a material that is capable of being punctured with a needle 52 whereupon the needle 52 cuts through the membrane 50 and creates an access path through the membrane 50 into the fluid channel 38. The walls of the access path are forced apart by the presence of the needle 52 such that when the needle 52 is removed from the membrane 50, the access path resumes a closed position thereby preventing a flashback and/or leakage of the fluid contained in the infusion system 12.

Again referring to FIGS. 1 & 2, the patient's vascular system 18 is accessed as a probe 53 is inserted into the valve 24 whereupon the probe tip 54 is introduced into a flow path 44. Once the probe tip 54 is introduced into the flow path, the user and/or clinicians may access the patient's vascular system 18 through the infusion system 12.

Referring now to FIG. 2, the y-port device 10 is comprised of a first tubular member 26 having a first end 32 and a second end 34 and extending in a generally longitudinal direction 30. The first tubular member 26 is generally cylindrical but may include other hollow, tube-like configurations such as square tubing or multi-angular tubing. The first tubular member 26 comprises a rigid, plastic material but may include flexible, pliable or non-rigid materials as well such as nylon tubing, polyurethane tubing, surgical tubing or Teflon tubing. In one embodiment, the first tubular member 26 comprises polypropylene material and is rigid.

The first tubular member 26 further comprises a first end 32 with an inner diameter to accommodate the fitting of a valve 24. The inner diameter of the first end 32 is engineered to receive the valve 24 such that the valve 24 fits securely within the first end 32 in a fluidtight fashion. The valve 24 may be secured within the first end 32 by friction, by an adhesive and/or by a complimentary design wherein the valve 24 contains a feature that is complimented by a feature located on the interior surface of the first end 32 of the first tubular member 26 such that the valve 24 and the first end 32 are locked together in a fluidtight fashion.

The first tubular member 26 further comprises a second end 34. The second end 34 is located at the end opposite to the first end 32 and has an inner diameter engineered to support intravenous tubing 16 such that the intravenous tubing 16 is irreversibly supported by the inner walls of the second end 34 of the first tubular member 26 in a fluidtight fashion. The intravenous tubing 16 may be supported by friction, an adhesive and/or by a complimentary design wherein the outer surface of the intravenous tubing 16 contains a feature that is complimented by a feature located on the interior surface of the second end 34 of the first tubular member 26 such that the intravenous tubing 16 and the second end 34 are locked together in a fluidtight fashion.

The y-port device 10 further comprises a second tubular member 28. The second tubular member 28 is generally cylindrical but may include other hollow, tube-like configurations such as square tubing or multi-angular tubing. The second tubular member 28 comprises a rigid, plastic material but may include flexible, pliable or non-rigid materials such as nylon tubing, polyurethane tubing, surgical tubing or Teflon tubing. In one embodiment, the second tubular member 28 comprises polypropylene material and is rigid. The second tubular member 28 further comprises a first end 37 with an inner diameter engineered to support intravenous tubing 16 such that the intravenous tubing 16 is irreversibly supported by the inner walls of the first end 37 of the second tubular member 28 in a fluidtight fashion. The intravenous tubing 16 may be supported by friction, an adhesive and/or by a complimentary design wherein the outer surface of the intravenous tubing 16 contains a feature that is complimented by a feature located on the interior surface of the first end 37 of the second tubular member 28 such that the intravenous tubing 16 and the first end 37 are locked together in a fluidtight fashion.

The second tubular member 28 further comprises a second end 39. The second end 39 forms a junction 36 with the first tubular member 26 and the second tubular member 28 intersects the first tubular member 26 an angle θ of 90° or greater. For example, in one embodiment the second tubular member 28 intersects the first tubular member 26 at an angle θ of 120°. In another embodiment, the second tubular member 28 intersects the first tubular member 26 at an angle θ that provides a continuous fluid channel 38 through the interior of the y-port device 10. In another embodiment, the angle θ is selected to provide adequate clearance between the first end 32 of the first tubular member 26 and the first end 37 of the second tubular member 28 such that a clinician may access the valve 24 without being encumbered by the position of the second tubular member 28.

The junction 36 between the first tubular member 26 and the second tubular member 28 may be formed by various plastic molding techniques including plastic injection molding and compression molding, and/or by various plastic joining techniques including heated tool, hot gas, laser welding, mechanical fastening and chemical bonding.

The y-port device comprises a valve 24, as previously discussed. The valve 24 is positioned within the first tubular member 26 such that a first end 40 of the valve 24 corresponds to the first end 32 of the first tubular member 26. The second end 42 of the valve 24 is angled at an angle θ′ generally corresponding to the angle θ of the intersecting second tubular member 28. For example, in one embodiment, the junction 36 is at an angle θ of 120° and the second end 42 of the valve 24 is at an angle θ′ of 120°. In another embodiment, the junction 36 is at an angle θ that provides a continuous fluid channel 38 through the interior of the y-port device 10 and the second end 42 of the valve 24 is at an angle θ′ which is equal to angle θ.

The second end 42 of the valve 24 abuts the fluid channel 38 such that there is no recessed cove or gap between the fluid channel 38 and the second end 42 of the valve 24. The second end 42 of the valve 24 extends up to the fluid channel 38, but does not extend into the fluid channel 38. The flow path 44 runs through the fluid channel 38 and is in direct fluid communication with the second end 42 of the valve 24 such that the second end 42 comprises a portion of the perimeter of the fluid channel 38, but does not disrupt and/or divert the flow path 44. For example, in one embodiment a fluid enters the fluid channel 38 through the second tubular member 28 and continues through the fluid channel 38 bypassing the valve 24 and following the flow path 44 through the interior of the y-port device 10, through the second end 34 of the first tubular member 26 and out of the y-port device 10. In this same embodiment, the fluid bypasses the second end 42 of the valve 24 without changing its velocity or flow pattern due to the presence of the valve 24. The interface between the second end 42 of the valve 24 and the fluid in the fluid channel 38 results in a uniform flow pattern and velocity of the fluid through the fluid channel 38 of the y-port adapter 10.

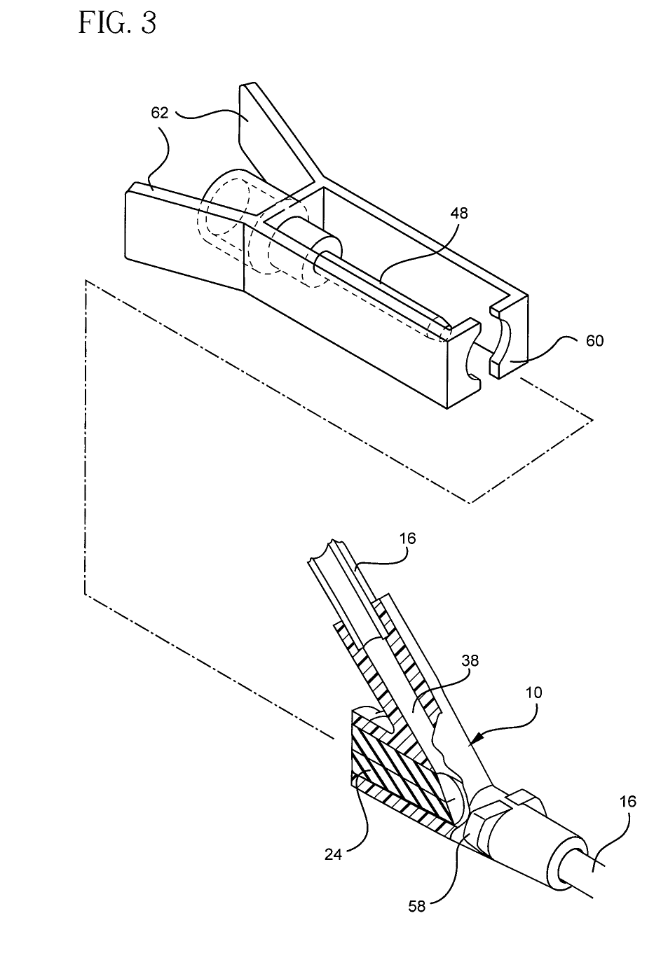

Referring now to FIGS. 2-4, the valve 24 may include a split septum 46. The valve 24 may include a solid or semi-solid plug that is split in such a way as to allow a probe 53 access to the fluid channel 38 through the septum split 46 (discussed above in detail). The first end 40 of the valve 24 may extend to the rim of the first end 32 of the first tubular member 26 such that the first end 40 of the valve 24 may be cleaned and/or sterilized prior to insertion of a probe 53. For example, in one embodiment the first end 40 of the valve 24 is sterilized with an alcohol swap prior to the introduction of a blunt, male Luer into the split 46 of the valve 24. In another embodiment, the first end 40 of the valve 24 is sterilized with an alcohol swap prior to puncturing the membrane 50 of the valve 24 with a hypodermic needle 52.

The first end 32 of the first tubular member 26 may be modified to include a feature 58 for attaching additional components of the infusion system. For example, in one embodiment the feature 58 is male threads adapted to compatibly receive female threads incorporated into one end of a probe 53, such as a male Luer. In another embodiment, the feature 58 is a raised portion of the outer surface of the first tubular member 26 wherein the raised portion is designed to receive a complementary clip 60 as incorporated into a probe 53, such as a male Luer. In this same embodiment, the complementary clip 60 engages the external feature 58 in a reversible manner such that the complementary clip 60 includes a pressure sensitive clasp or pinching mechanism 62 whereby a user and/or clinician may pinch the mechanism 62 to release the complementary clip 60 from the external feature 58. It is also anticipated that the first end 37 of the second tubular member 28 and the second end 34 of the first tubular member 26 may also be modified to include a feature 58 for attaching additional components of the infusion system 12 as described above.

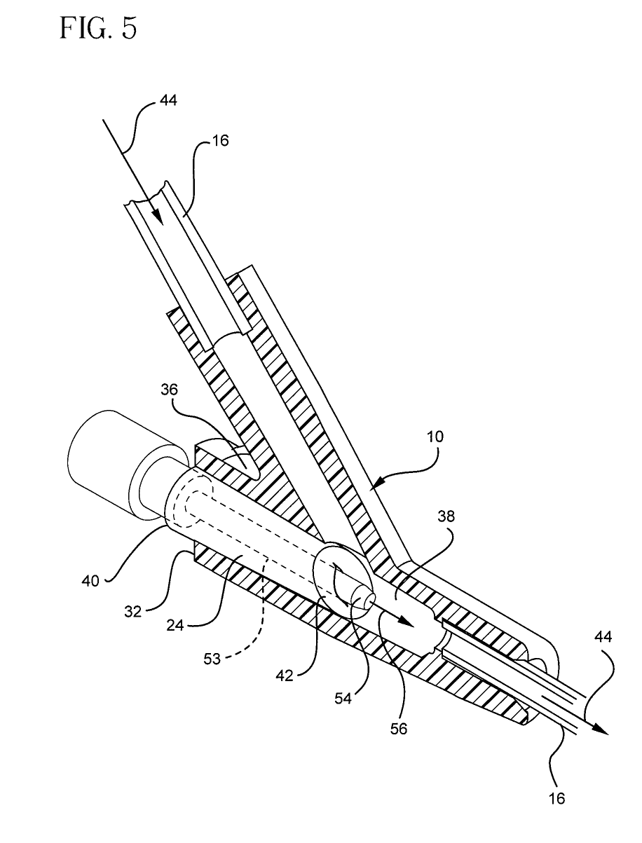

Referring now to FIG. 5, the valve 24 is positioned such that upon penetration of a probe 53 the probe tip 54 exits the second end 42 of the valve 24 directly into the fluid channel 38 permitting a fluid 56 to be infused directly into the flow path 44 thereby ensuring that all of the intended fluid 56 is infused into the infusion system 12 and into the patient's vascular system 18 (not shown). The fluid channel 38 is configured such that the inner diameter of the fluid channel 38 is greater than the outer diameter of the probe 53 such that the probe 53 may enter the fluid channel 38 without blocking the flow path 44.

The present invention may be embodied in other specific forms without departing from its structures, methods, or other essential characteristics as broadly described herein and claimed hereinafter. For example, the present invention may be incorporated into any system comprising a valve and a fluid channel where undesirable stagnation or concentration of one fluid within another fluid occurs. For example, the present invention may be applied in a coolant system where a fluid with a first temperature is released into a fluid with a second temperature by means of a valve, wherein a concentration or stagnation of the first fluid within the second fluid, due to the recessed positioning of the valve, is undesirable. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A y-port device comprising:
a first tubular member having a first end, a second end and dead space, the first tubular member extending in a generally longitudinal direction;
a second tubular member intersecting the first tubular member at an angle of 90° or greater, and forming a junction with the first tubular member;
a valve positioned within the first tubular member to eliminate the dead space while maintaining a fluid channel between the second tubular member and the first tubular member.

2. A y-port device as defined in claim 1, wherein the valve has a first end corresponding to the first end of the first tubular member and a second end corresponding to the junction.

3. The y-port device as defined in claim 1, wherein the valve is a septum.

4. A y-port device as defined in claim 3, wherein the second end of the septum is angled at an angle generally corresponding to the angle of intersection between the first tubular member and the second tubular member.

5. A y-port device as defined in claim 1, wherein the valve is penetrable by a probe.

6. The y-port device as defined in claim 1, wherein an interface between the valve and the fluid channel eliminates a concentrating of a first fluid within first tubular member of the y-port adapter.

7. The y-port device as defined in claim 3, wherein the septum is oriented such that upon penetrating the septum with a probe, the probe is positioned in the fluid channel whereby a fluid from the probe is directed into a flow path of the fluid channel.

8. The y-port device as defined in claim 7, wherein the flow path comprises one direction of flow.

9. The y-port device as defined in claim 5, wherein the first tubular member has an inner diameter and the probe has an outer diameter such that the inner diameter of the first tubular member is greater than the outer diameter of the probe.

10. The y-port device as defined in claim 5, wherein an outer surface of the first tubular member comprises an external feature to couple the probe to the first tubular member.

11. A method of eliminating aberrant currents within a y-port device comprising the step of positioning a valve within a first portion of the y-port device to eliminate a dead space within the first portion, the valve being juxtapose to a fluid channel of the y-port device such that an end surface of the valve creates an interface with a fluid in the fluid channel wherein the interface eliminates aberrant currents within the fluid channel.

12. The method of claim 11, wherein the valve is a septum.

13. The method of claim 13, wherein the septum is positioned within the first portion of the y-port device such that upon pushing a probe through the septum, the probe is positioned in the fluid channel whereby a fluid from the probe is directed into a flow path of the fluid channel.

14. The method of claim 13, wherein the flow path comprises one direction of flow.

15. The method of claim 11, wherein the first portion of the y-port device has an inner diameter and the probe has an outer diameter such that the inner diameter of the first portion is greater than the outer diameter of the probe.

16. The method of claim 11, wherein the first tubular member comprises an external feature to aid in coupling the probe to the first tubular member.

17. A y-port device system comprising:
a first tubular member having a first end, a second end and an opening, the opening being in a first plane, and the first tubular member extending in a longitudinal direction perpendicular to the first plane;
a second tubular member intersecting the first tubular member at a first angle of greater than 0° and less than 180°, and forming a junction with the first tubular member;
a valve having a first end surface and a second end surface, the first end surface corresponding to the first end of the first tubular member and being in the first plane, the second end surface corresponding to the junction and being angled relative to the first plane at a second angle of greater than 0° and less than 180°, the valve being located within the first tubular member and eliminating a dead space within the first tubular member between the first end and the junction, while maintaining a fluid channel between the second tubular member and the first tubular member, wherein the valve means is penetrable by a probe for accessing the fluid channel.

18. The system of claim 17, wherein an interface between the second end surface of the valve and the fluid channel eliminates aberrant currents within the fluid channel.

19. The system of claim 18, wherein the valve is oriented such that upon pushing the probe through the valve, a tip of the probe is positioned in the fluid channel whereby a fluid from the probe is directed into a flow path of the fluid channel.

20. The system of claim 19, wherein the flow path comprises one direction of flow.
